(19) United States
(12) Patent Application Publication
(10) Pub. No.: US 2025/0363255 A1
(43) Pub. Date: Nov. 27, 2025

(54) SYSTEM AND METHOD OF DETERMINING COMPOSITION OF A DRUG

(71) Applicant: B. G. NEGEV TECHNOLOGIES AND APPLICATIONS LTD., AT BEN-GURION UNIVERSITY, Beer-Sheva (IL)

(72) Inventors: Bracha SHAPIRA, Beer Sheva (IL); Eyal MAZUZ, Beer Sheva (IL); Lior Shimon ROKACH, Beer Sheva (IL); Guy SHTAR, Beer Sheva (IL); Adi JABARIN, Beer Sheva (IL); Shimon BEN-SHABAT, Beer Sheva (IL)

(73) Assignee: B. G. NEGEV TECHNOLOGIES AND APPLICATIONS LTD., AT BEN-GURION UNIVERSITY, Beer-Sheva (IL)

(21) Appl. No.: 19/290,537

(22) Filed: Aug. 5, 2025

Related U.S. Application Data
(63) Continuation of application No. PCT/IL2024/050133, filed on Feb. 5, 2024.
(60) Provisional application No. 63/443,402, filed on Feb. 5, 2023.

(51) Int. Cl. G06F 30/12; G16C 20/50

(52) U.S. Cl. CPC G06F 30/12; G16C 20/50

(57) ABSTRACT

A system and method of designing a drug by at least one processor may include obtaining a molecule string data element, representing ad-hoc structure of a molecule. The molecule string may include at least one token, representing (i) indication of a beginning of the molecule string, (ii) one or more components of the molecule, and/or (iii) relation between components of the molecule. The at least one processor may apply an embedding algorithm on the molecule string, to obtain an embedding vector, representing the ad-hoc structure of the molecule in an embedding space, and apply a pretrained transformer-based decoder model on the embedding vector, to select a subsequent token from a predetermined set of tokens; append the predicted token to the molecule string; and, following identification of occurrence of an end condition, append a token representing end of the molecule string, to determine composition of the drug.

### CROSS-REFERENCE TO RELATED APPLICATIONS

This application is a National Phase Application of PCT International Application No. PCT/IL2024/050133, having International Filing Date of Feb. 5, 2024, titled “SYSTEM AND METHOD OF DETERMINING COMPOSITION OF A DRUG”, which claims the benefit of priority of U.S. Patent Application No. 63/443,402, titled: “NCE GENERATION USING RL AND NLP METHODS”, filed Feb. 5, 2023, both hereby incorporated by reference in their entirety.

### FIELD OF THE INVENTION

The present invention relates generally to drug discovery. More specifically, the present invention relates to systems and methods of determining composition of a drug.

### BACKGROUND OF THE INVENTION

A major challenge in drug discovery is designing drugs with the desired properties. The chemical space of potential drug-like molecules is between 10²³ to 10⁶⁰, of which about 10⁸ molecules are synthesized. Additionally, the average cost of developing a new drug is one to two billion US dollars, and the average development time is 13 years. Traditionally, chemists and pharmacologists use their intuition and expertise to identify new molecules. While Lipinski's “rule of five” may reduce the number of possible drug-like molecules, the search space remains large. In order to narrow the space further, high-throughput screening (HTS) is used; however, the task remains daunting.

In recent years, there have been many attempts to use deep learning, particularly generative models, for drug design. However, the task of generating optimized and valid molecules using computational methods remains challenging due to the large search space and the small number of labeled samples.

There have been several attempts to use Simplified Molecular-Input Line-Entry System (SMILES) strings as a representation for molecules. For example, some works tried using generative models based on SMILES strings for the molecule generation task. However, the proposed methods only managed to generate a low percentage of molecules that were considered valid due to the complicated grammatical rules of SMILES.

Recently, the use of reinforcement learning (RL) has gained attention due to its ability to solve a wide range of problems such as playing the game of Go and operating machines. RL systems excel in these tasks thanks to their ability to make sequential decisions and maximize defined long-term rewards; this allows for the direct optimization of desirable new drug properties that are not derived from the model itself when using generative models such as recurrent neural networks (RNNs). In subsequent studies, RL optimization was incorporated into SMILES generation methods to generate molecules with desired properties, such as high IC50 values for JAK2, using a RNNs. Such optimization is technically challenging, since it tends to cause the model to converge toward a set of primarily invalid molecules, since RNNs cannot handle long sequences.

### SUMMARY OF THE INVENTION

To improve the rate of valid molecules generated, some studies constrained the input of generative models when producing molecules by forcing the model to adhere to certain rules when generating molecules. Some studies proposed the use of variational autoencoders (VAEs) to generate valid molecules by learning the distribution of a latent space and sampling from it, instead of sequentially generating the molecule token by token. However, the validity rate of these methods was relatively low. These results could be explained by the lower validity rate obtained in those studies for unseen molecules compared to known ones.

To address this issue, the inventors proposed the junction tree variational autoencoder (JTVAE), representing molecules as junction trees in order to encode the sub-spaces of the molecular graph. This configuration may allow the decoder to generate valid molecules by utilizing only valid components, while considering how they interact.

The inventors have proposed a new RL-based method to generate molecules with desired properties, which overcomes the problem of generating valid molecules with desired properties. The inventors use a transformer-based architecture, utilizing SMILES string representations in a two-stage approach.

The present invention may include a synergistic approach that utilizes both transformer models and reinforcement learning together, for molecule graph generation. Embodiments of the invention may thus provide a practical application for improving the technology of drug design.

In a first stage, the model may learn to embed discrete string representations in a vector space. Then, in the second stage, the model may optimize the vector space in order to generate molecules with the desired properties, such as QED (quantitative estimate of drug-likeness) or pIC50.

The use of an attention mechanism allows embodiments of the invention to gain an understanding of the underlying chemical rules that make a valid molecule by performing a simple language modelling task, using just a small amount of data. Then, the understanding gained regarding those rules, along with policy gradient RL, may be used to generate molecules with the desired properties. As elaborated herein, the inventors evaluated their model on multiple datasets with various properties on the tasks of molecule generation and optimization for the desired properties and compared it to several state-of-the-art approaches that use different representations and techniques for molecule generation.

Embodiments of the invention may include a method of designing a drug by at least one processor. Embodiments of the method may include an iterative, transformer-based generative process, wherein in each iteration the at least one processor may be configured to obtain a molecule string data element, representing ad-hoc structure of a molecule, where the molecule string contains at least one token, and where the token represents at least one of (i) an indication of a beginning of the molecule string, (ii) one or more components of the molecule, and/or (iii) relation between components of the molecule. In each iteration, the at least one processor may apply an embedding algorithm on the molecule string, to obtain an embedding vector, representing the ad-hoc structure of the molecule in an embedding space; applying a pretrained transformer-based decoder model on the embedding vector, to select a subsequent token from a predetermined set of tokens; and append the predicted token to the molecule string. Following identification of occurrence of an end condition, the at least one processor may append a token representing end of the molecule string, thereby finalizing the molecule string, and determining composition of the drug.

Additionally, or alternatively, the at least one processor may be configured to implement an iterative Reinforcement Learning (RL) process, concurrent with, or controlling the iterative transformer-based generative process. In each iteration of the RL based process, the at least one processor may analyze the finalized molecule string, to obtain a reward value; retrain the decoder model based on the obtained reward value; and reinvoke the generative process, to produce another finalized molecule string, until a predetermined condition is satisfied.

According to some embodiments, the at least one processor may be configured to analyze the finalized molecule string by calculating a 3-Dimensional (3D) model representing a 3D structure of an underlying molecule based on the finalized molecule string; analyzing the 3D model to obtain values of one or more metrics of molecule properties; and calculating the reward value based on the one or more metrics of molecule properties. In such embodiments, the transformer-based generative process may be reinvoked until a predetermined condition on the one or more metrics of molecule properties is satisfied, as elaborated herein.

Additionally, or alternatively, the at least one processor may be configured to analyze the 3D model by applying a validation algorithm on the 3D model, to obtain a molecule-specific validity score of the underlying molecule. In such embodiments, the metric of molecule properties may include the molecule-specific validity score.

Additionally, or alternatively, the at least one processor may be configured to analyze the 3D model by applying the validation algorithm on a plurality of 3D models, originating from a respective plurality of finalized molecule strings, to obtain a respective plurality of molecule-specific validity scores; and based on the plurality of molecule-specific validity scores, calculating an agent validity score, representing a percentage of valid finalized molecule strings from the plurality of finalized molecule strings. In such embodiments, the metric of molecule properties may include the agent validity score.

Additionally, or alternatively, the at least one processor may be configured to analyze the 3D model by applying a Quantitative Estimation of Drug-Likeness (QED) algorithm on the 3D model, to obtain a molecule-specific QED score of the underlying molecule. In such embodiments, the metric of molecule properties may include the molecule-specific QED score.

Additionally, or alternatively, the at least one processor may be configured to analyze the 3D model by applying a Synthetic Accessibility Score (SAS) algorithm on the 3D model, to obtain a molecule-specific SAS score of the underlying molecule. In such embodiments, the metric of molecule properties may include the molecule-specific SAS score.

Additionally, or alternatively, the at least one processor may be configured to analyze the 3D model by: invoking the generative process a plurality of times, to obtain a plurality of finalized molecule strings; and based on the member tokens of the plurality of finalized molecule strings, calculating a molecule diversity score, representing a diversity among the plurality of finalized molecule strings. In such embodiments, the metric of molecule properties may include the molecule diversity score.

Additionally, or alternatively, the at least one processor may be configured to analyze the finalized molecule string by: applying a pretrained Machine Learning (ML) based classification model on the finalized molecule string, to predict a value of efficacy of a respective molecule in treatment of a predetermined medical condition; and calculating the reward value based on the predicted efficacy value. The at least one processor may subsequently reinvoke the generative process until a predetermined condition on the predicted efficacy value is satisfied.

According to some embodiments, the transformer-based decoder model may include a one or more (e.g., a plurality) of attention heads. At least one (e.g., each) attention head may be pretrained to provide a distribution of probabilities for selecting specific tokens of the set of tokens, based on different locations in the molecule string.

According to some embodiments, the decoder model may be configured to select the subsequent token based on the distribution of probabilities provided by the plurality of attention heads. The at least one processor may be configured to retrain the decoder model comprises adjusting the distribution of probabilities based on the obtained reward value.

Embodiments of the invention may include a system for designing a drug. Embodiments of the system may include a non-transitory memory device, where modules of instruction code are stored, and at least one processor associated with the memory device, and configured to execute the modules of instruction code.

Upon execution of said modules of instruction code, the at least one processor may be configured to: obtain a molecule string data element, representing ad-hoc structure of a molecule, wherein said molecule string contains at least one token, representing (i) indication of a beginning of the molecule string, (ii) one or more components of the molecule, or (iii) relation between components of the molecule; apply an embedding algorithm on the molecule string, to obtain an embedding vector, representing the ad-hoc structure of the molecule in an embedding space; apply a pretrained transformer-based decoder model on the embedding vector, to select a subsequent token from a predetermined set of tokens; append the predicted token to the molecule string; and following identification of occurrence of an end condition, append a token representing end of the molecule string, thereby finalizing the molecule string and determining composition of the drug.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a block diagram, depicting a computing device which may be included in a system for designing a drug, according to some embodiments;

FIGS. 2A and 2B are schematic diagrams, which depict an overview of a training process of a system 100, also referred to herein as “TAIGA” for designing a drug, according to some embodiments of the invention;

FIG. 3 is a table (Table 1), presenting overall training data statistics that were experimentally obtained by embodiments of the invention;

FIG. 4 is a table (Table 2), presenting comparison of performance on a property optimization task, experimentally obtained by embodiments of the invention;

FIG. 5 is a table (Table 3), presenting the results of the top 3 molecules generated with different datasets in terms of their respective QED scores, and the aggregate results of the validity, QED, and diversity, as experimentally obtained by embodiments of the invention;

FIG. 6 is a table (Table 4), presenting performance of a property optimization task, as experimentally obtained by embodiments of the invention;

FIG. 7 is a table (Table 5), presenting a comparison of the results for the main metrics of TAIGA, with and without the RL stage, as experimentally obtained by embodiments of the invention;

FIG. 8 is a table (Table 6), presenting cross-dataset novelty scores of the molecules generated, as experimentally obtained by embodiments of the invention;

FIG. 9 is a chart showing experimental results of TAIGA's QED performance as a function of molecule validity rate;

FIG. 10 is a block diagram, depicting a system (“TAIGA”) for designing a drug, according to some embodiments of the invention;

FIG. 11 is a block diagram, depicting a transformer-based agent that may be included in a system for designing a drug, according to some embodiments of the invention; and

FIGS. 12A and 12B jointly depict a flow diagram of a method of designing a drug, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

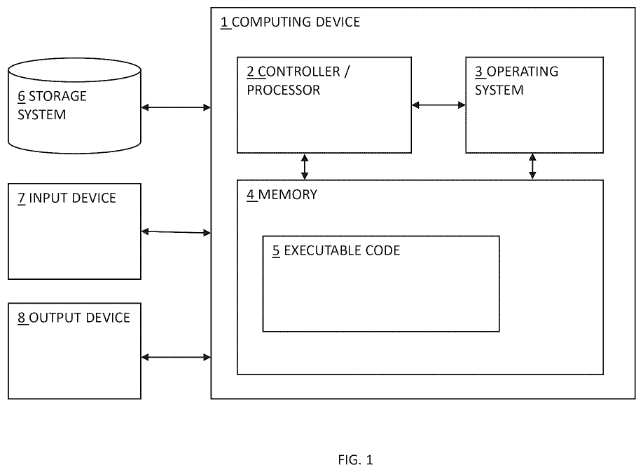

Reference is now made to FIG. 1, which is a block diagram depicting a computing device, which may be included within an embodiment of a system for TBD, according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task, or script. Executable code 5 may be executed by processor or controller 2 possibly under control of operating system 3. For example, executable code 5 may be an application that may TBD as further described herein. Although, for the sake of clarity, a single item of executable code 5 is shown in FIG. 1, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data TBD may be stored in storage system 6 and may be loaded from storage system 6 into memory 4 where it may be processed by processor or controller 2. In some embodiments, some of the components shown in FIG. 1 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components, or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

The term neural network (NN) or artificial neural network (ANN), e.g., a neural network implementing a machine learning (ML) or artificial intelligence (AI) function, may be used herein to refer to an information processing paradigm that may include nodes, referred to as neurons, organized into layers, with links between the neurons. The links may transfer signals between neurons and may be associated with weights. A NN may be configured or trained for a specific task, e.g., pattern recognition or classification. Training a NN for the specific task may involve adjusting these weights based on examples. Each neuron of an intermediate or last layer may receive an input signal, e.g., a weighted sum of output signals from other neurons, and may process the input signal using a linear or nonlinear function (e.g., an activation function). The results of the input and intermediate layers may be transferred to other neurons and the results of the output layer may be provided as the output of the NN. Typically, the neurons and links within a NN are represented by mathematical constructs, such as activation functions and matrices of data elements and weights. At least one processor (e.g., processor 2 of FIG. 1) such as one or more CPUs or graphics processing units (GPUs), or a dedicated hardware device may perform the relevant calculations.

The inventors demonstrated the model's ability to generate a high percentage of valid molecules, in relation to currently available methods of drug design. Additionally, unlike previous research that only focuses on the top molecules generated, the inventors show the model's ability to generate a large number of molecules with a high mean QED, which defines how drug-like a molecule is, while maintaining a low Synthetic Accessibility Score (SAS), a theoretic score of how hard it is to synthesize the molecule.

As elaborated herein, in the task of optimizing a biological property (i.e., IC50), embodiments of the invention may be capable of improving existing molecules and generating molecules with the desired biological properties.

The inventors have contributed by introducing an RL-based system and method for designing a drug, also referred to herein as “TAIGA”. As shown herein, TAIGA may utilize a transformer architecture to generate novel and diverse molecules, e.g., for use in the pharmaceutical industry.

The inventors demonstrated that the use of an attention mechanism combined with policy gradient RL can overcome the existing challenges of generating valid molecules represented as SMILES strings.

The inventors performed extensive experiments using several datasets with a range of properties and multiple metrics to evaluate the performance of our method's components.

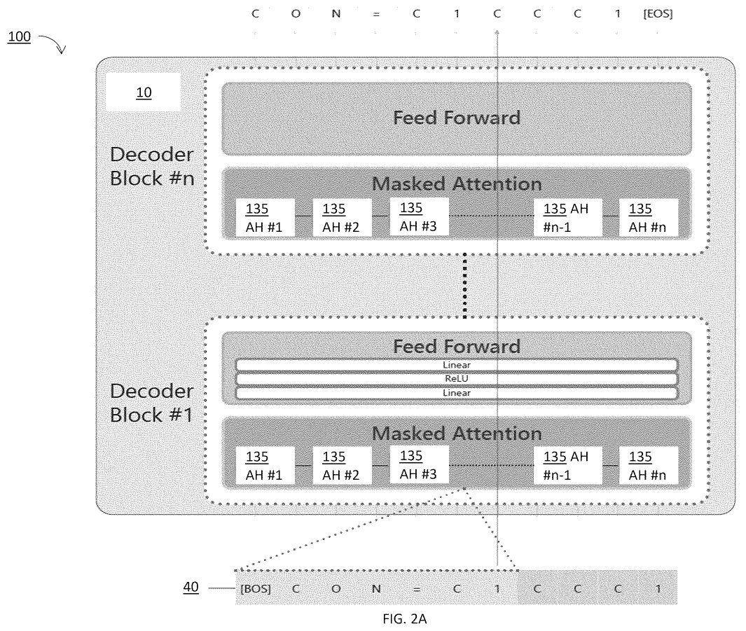

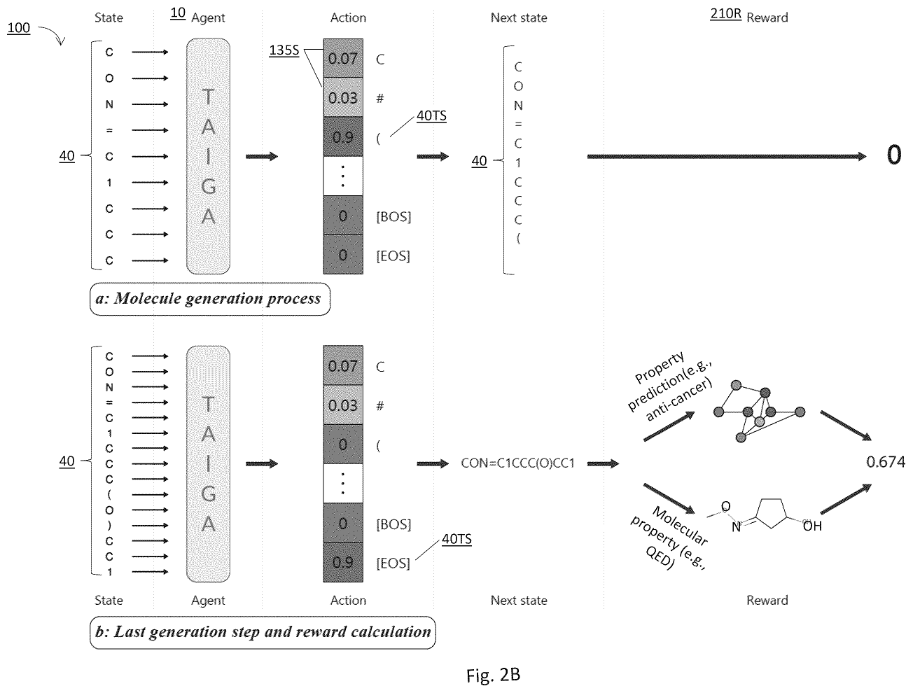

Reference is made to FIGS. 2A and 2B, which depict an overview of a training process of a system 100, also referred to herein as “TAIGA” for designing a drug, according to some embodiments of the invention.

As shown in FIG. 2A, during a first stage system 100 may train transformer-based generator model 10, also referred to herein as “agent 10”, on a language-modelling task of predicting the next token. In this example, the last input token was ‘1’, and the transformer-based generator model 10 selects 40TS, or predicts a subsequent token 40T in the sequence, in this case: ‘C’. According to some embodiments, agent 10 may be an auto-regressive model, meaning that it may only attend to previously selected tokens 40T. transformer-based generator model 10 (Agent 10) may include a plurality of attention heads, allowing it to perform selection 40TS or prediction of tokens 40T in parallel.

As shown in FIG. 2B (panel a), agent 10 may receive the ad-hoc molecule string 40 (e.g., SMILES) and predict or select 40TS the next token 40T by sampling from the output distribution of the attention heads 135. The agent may then appended the selected token 40TS (now 40T) to the ad-hoc molecule string 40, to update the content of string 40, also referred to herein as a “state” of molecule string 40. The terms “molecule string 40” and “state 40” may be used interchangeably. system 100 may attribute a null (zero) reward value 210R to molecule string 40, as long as molecule 40 is not yet finalized.

As shown in FIG. 2B (panel a), agent 10 may complete the molecule string 40 generation process by predicting an EOS token thereby producing a finalized molecule string 40F. System 100 may assign a reward 210R other (e.g., greater) than zero to finalized molecule string 40.

In some embodiments, system 100 may utilize currently available libraries (e.g., such “RDKit”), to create a three-dimensional (3D) model of the underlying molecule, and then calculate reward 210R based on properties of the 3D model, as elaborated herein.

Additionally, or alternatively, system 100 may infer a currently available property predictor (e.g. “Chemprop”) on molecule string 40, to predict a property of the underlying, represented molecule (e.g., having anti-cancerous properties), and generate reward 210R based on the predicted property.

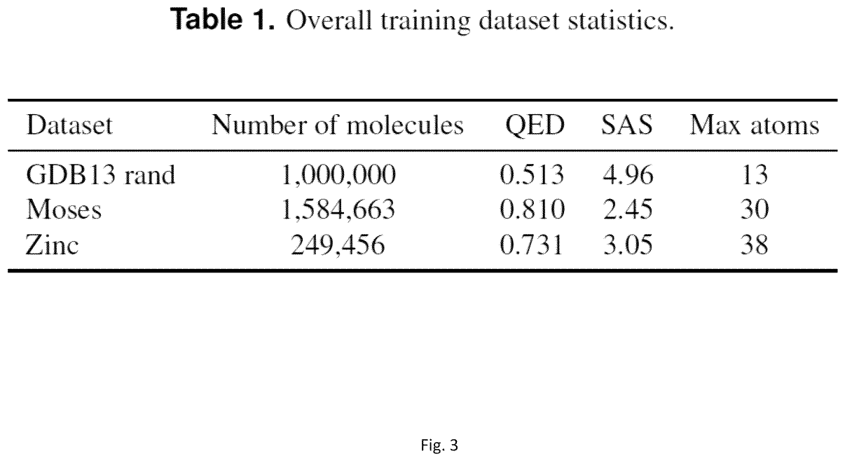

Reference is made to FIG. 3, which depicts a table (Table 1), presenting overall training data statistics.

In this section, to evaluate TAIGA's performance, the inventors validate it on 2 common tasks: molecule generation and molecule optimization, for the molecule generation task the inventors compare TAIGA to the performance of several SOTA baselines using multiple datasets and metrics.

Data: The inventors used three datasets in our experiments: the Moses, Zinc, and GDB13 datasets.

The Moses dataset consists of 1.6M training set molecules extracted from the Zinc clean lead collection.

The Zinc dataset consists of 250K molecules extracted from the Zinc database.

The GDB13 rand dataset consists of 1M random molecules extracted from the larger GDB13 database.

These datasets differ from one another in terms of the number and type of molecules included. Experimenting on these different datasets allows us to demonstrate the generalization ability of the methods evaluated.

Baselines: The inventors compared their method to various approaches: (1) GCPN, a method that uses proximal policy optimization (PPO) on molecular graphs, with the addition of adversarial loss; (2) JTVAE, a method which uses the junction tree algorithm to generate molecular graphs with autoencoders; (3) MolGPT, a method that generates SMILES using transformer architecture only; (4) MolGAN, a method that generates molecular graphs using GANs and DDPG; (5) MolDQN, a method that works on molecular graphs using Q-learning; (6) GraphDF a discrete normalizing flow method; and (7) an LSTM version of their method which also uses policy gradient RL, similar to. The inventors ran all models with their released code, optimizing for the same target property (i.e., QED), each with its respective reward function, as described in the original papers, on the same hardware containing one TITAN RTX GPU, 60 GB RAM, and an eight-core CPU.

Model Configuration: TAIGA may include four decoder layers, each with eight attention heads, a feedforward size of 1024, and an initial embedding size of 512. The model was trained for three epochs on the language modeling task and 100 steps, with each step averaging 500 episodes (molecules) in the RL stage. The inventors use the RL algorithm with a discount factor of 0.99 and a maximum sequence size of 150 characters. The LSTM-PG uses the same hyperparameters as TAIGA for all experiments.

In one experiment, the inventors designed the following reward function for their model:

$$R ( {s}_{T} ) = \{ \begin{matrix}10 * QED ( {s}_{T} ) , & if {s}_{T} is valid molecule \\ 0 , & otherwise\end{matrix}$$

The inventors utilized the following metrics to evaluate the methods:
Validity: the percentage of valid molecules a model is able to generate;
Novelty: the percentage of molecules generated that do not appear in the training data;
Diversity: the percentage of unique molecules the model can generate;
Quantitative Estimation of Drug-Likeness (QED), the geometric mean of eight common molecule properties that can estimate how well the molecule behaves in the human body; and
Synthetic Accessibility Score (SAS), an estimation of the how easy it is to synthesize a molecule by calculating the fragment score penalized by the complexity of the molecule.

The inventors calculated the QED and SAS metrics after removing all of the invalid molecules from the set of generated molecules. For all methods, the inventors generated the molecules after the optimization stage. For each method, the inventors generated 25K molecules to calculate the metrics.

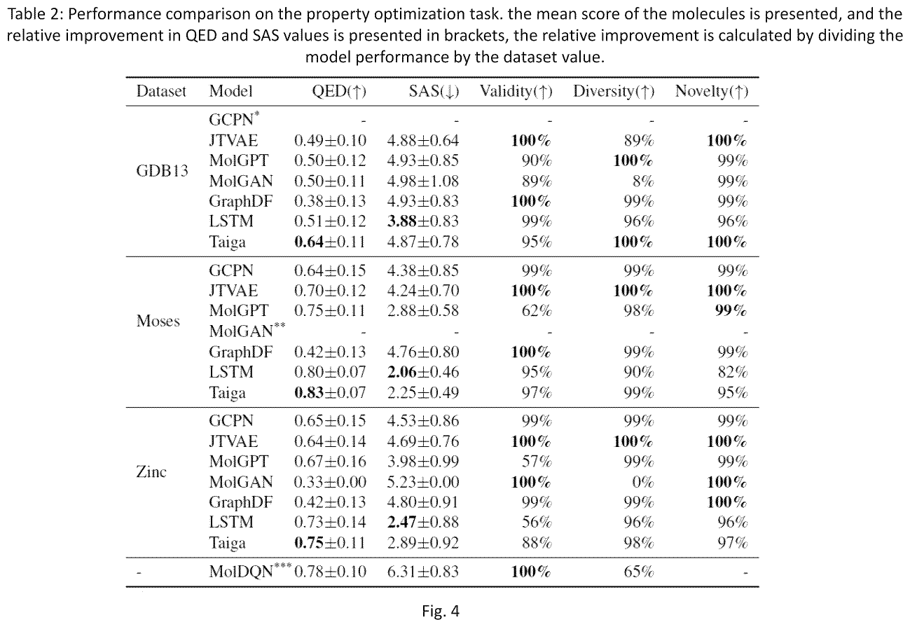

Reference is made to FIG. 4, which depicts a table (Table 2), comparing performance of property optimization, as experimentally obtained by embodiments of the invention.

Table 2 presents the results for molecule generation and optimization across all models and datasets. The mean score of the molecules is presented, and the relative improvement in QED and SAS values is presented in brackets, the relative improvement is calculated by dividing the model performance by the dataset value.

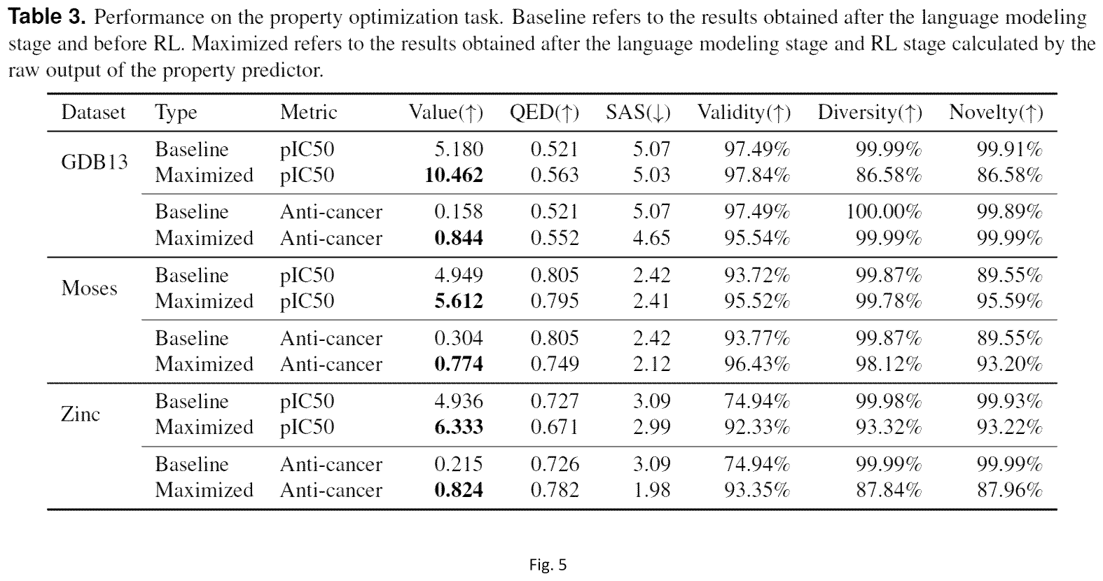

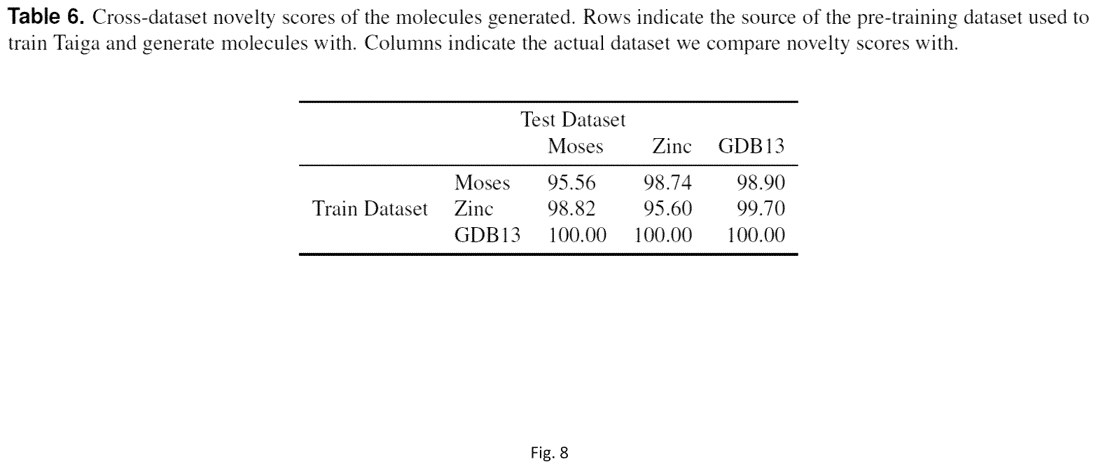

Reference is also made to FIG. 5, which depicts a table (Table 3). Table 3 (and FIG. 8) shows the results of the top-3 molecules generated with each dataset in terms of their respective QED scores, and the aggregate results of the validity, QED, and diversity, as experimentally obtained by embodiments of the invention.

As can be seen in Table 2, on the GDB13 dataset, which has a lower mean QED than the other datasets (see Table 1), TAIGA is the only method that was able to obtain QED scores higher than the dataset's mean QED score. Due to the fact that the GDB13 1m rand dataset is a random subset of the entire GDB13 dataset and is not preprocessed to contain lead-like molecules, thus making it more challenging for optimization and reaching good performance. This dataset also achieved the best scores in terms of novelty and diversity while maintaining a high validity score, this means that the molecules generated are not only valid but high novelty score means the inventors have higher chance to come across a lead molecule (since high novelty mean it doesn't overfit and generates molecules from the training set). As seen in Table 3, the inventors' method excels and is the only one that generated molecules with a QED value above 0.9. When looking at the SAS score of the best molecules in terms of the QED, the inventors find mixed results; this most likely occurred, because none of the methods directly tries to optimize the SAS score, and therefore when a model generates molecules, it generates compounds that are more complex and have a higher QED score but are harder to synthesize.

Of the three examined datasets, TAIGA achieved the best optimization results with the Moses dataset, while still maintaining a high value for validity, diversity, and novelty metrics averaging around 97%. Compared to graph-based methods like JTVAE and GCPN, which represent molecules as complex graphs to generate a high rate of valid molecules, the inventors' method achieved comparable results on the diversity and novelty metrics and was not far behind on the validity metric meaning that SMILES-based method can generate high percentage of valid molecules. On all metrics, the inventors' method performed the same or better than SMILES-based methods such as MolGPT and LSTM-PG. In Table 3, the inventors can see that TAIGA generated molecules with a higher QED value than that obtained by graph-based methods and other SMILES-based methods, while LSTM-PG did manage to create a high amount of valid molecules, it wasn't able to improve the metric it was optimized for which means that it can't generate both optimized and valid molecules which further emphasizes the limitation of LSTM-based method in comparison to Transformers.

On the Zinc dataset (see Table 2), most methods generated molecules with an average QED value similar to the dataset mean and a high SAS score, but TAIGA generated molecules with a QED value higher than the mean. Although graph-based methods such as GCPN and JTVAE were able to achieve a higher value on the validity metric than SMILES-based methods with this dataset, the inventors can see that TAIGA's validity scores are higher than those of MolGPT and LSTM-PG. In Table 3, the inventors can see that TAIGA and JTVAE obtained the best QED scores on the top molecules, while other methods such as LSTM-PG, GCPN, and MolGAN failed to obtain good QED scores for top molecules. In addition, in terms of the SAS score, TAIGA demonstrated superiority over the other methods, obtaining the best SAS scores for most molecules.

Compared to MolDQN, which achieved better QED scores than TAIGA on two of the three datasets without using a dataset during the training process, MolDQN achieved the lowest diversity scores out of all methods, which means that MolDQN is unable to generate a diverse set of molecules and generates the same molecule repeatedly. Similarly, it achieved the lowest SAS score out of all methods, thus generating molecules that are difficult to synthesize. This is due to the fact that MolDQN is a Q-learning algorithm, which at test time uses a greedy approach, and chooses actions based on the highest Q-values when generating molecules.

Aggregating the results, as seen in FIG. 2, shows TAIGA's superior performance across all datasets. TAIGA is located in the upper-right corner of the figure (high validity, high QED) and has high diversity (indicated by a larger circle). Most of the examined methods were unable to generate a large amount of valid molecules with high QED values; some methods (e.g., JTVAE) were able to achieve good validity and diversity scores but at the cost of degraded performance on the target properties that the model tried to optimize. On the other hand, TAIGA achieved better performance on the target task of optimizing for the desired property at the cost of a slightly lower validity rate.

In this subsection, the inventors evaluate TAIGA's ability to optimize biological properties with therapeutic function, which are harder to predict than QED; such a task requires additional supervised learners to predict molecular properties.

Data: The inventors used 2 datasets, the first is IC50 data extracted from the ChEMBL database21 and extracted all of the molecules that have exact pIC50 values, i.e., the inventors removed molecules for which only a range is available. pIC50 is the negative log of the IC50 value by using the following formula: 9-log10 (IC50). The inventors focused specifically on the BACE (Beta-secretase 1) protein. After filtering out 10,164 molecules, the inventors ended up with 9,331 samples with exact pIC50 values. The second is dataset of molecules that used in cancer treatments, the inventors collected around 400 molecules from various sources that had indication for some anti-cancer activity (FDA approval, clinical trials, etc.) and around 1000 molecules that are not known for treating cancer.

Model Configuration: The inventors used the same configuration for TAIGA as the one mentioned in the Molecule Generation Section. For the property prediction model the inventors utilize Chemprop, a message passing graph neural network (MPNN) since its ability to predict potential molecules. The inventors train the model with the default parameters the library provides.

The inventors used the following reward function for the IC50 task as in the equation below:

$$R ( {s}_{T} ) = \{ \begin{matrix}exp ( \frac{pIC 50}{3} ) , & if {s}_{T} is valid molecule \\ 0 , & otherwise\end{matrix} \qquad Eq . 2$$

For the anti-cancer prediction the inventors use the following reward function, as in the equation below:

$$R ( {s}_{T} ) = \{ \begin{matrix}Chemprop ( {s}_{T} ) , & if {s}_{T} is valid molecule \\ 0 , & otherwise\end{matrix} \qquad Eq . 3$$

where Chemprop (sT) is the raw output probability of the MPNN which ranges between 0 and 1 model raw output probabilities.

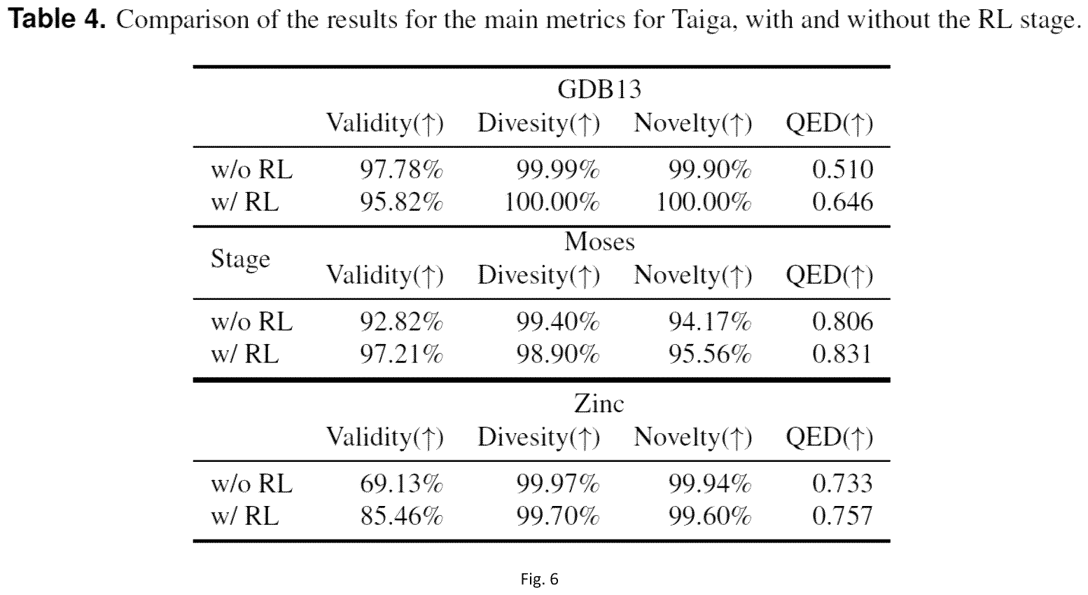

Reference is also made to FIG. 6, which depicts a table (Table 4). Table 4 shows performance of a property optimization task, experimentally obtained by embodiments of the invention. Baseline refers to the results obtained after the language modeling stage and before RL. Maximized refers to the results obtained after the language modeling stage and RL stage calculated by the raw output of the property predictor.

Results: The results presented in Table 4 demonstrate TAIGA's ability to maximize pIC50 values with the different datasets. The inventors can see that when using all of the datasets as baselines, TAIGA can be optimized for biological properties. On average, TAIGA increased the pIC50 value by 20% when converting to IC50 values; this is the equivalent of reducing the concentration by a factor of 3-5 for the same therapeutic effect. The inventors can see that the validity constraint of Eq. 2 helps maintain the same validity scores as the baseline. This prevents overfitting by generating random strings that can exploit the property predictor.

Looking at other metrics such as the QED or SAS, the inventors can see that TAIGA was able to generate molecules with improved pIC50 values and at the same maintained similar SAS and QED values to those of the baseline; this means that it did not only do the molecules have a better potential for treatment, they are also easy to synthesize and have drug-like properties. With two of the three datasets, TAIGA was able to keep generating a set of novel and diverse molecules after the RL stage, and on the Zinc and GDB dataset, the novelty and diversity scores decreased but by just a small margin. This means that TAIGA was able to generate a set of molecules with higher pIC50 values while ensuring that the molecules are different from each other and were not seen during the training process.

When generating molecules to have anti-cancer activity, the inventors also see that TAIGA can maximize and generate molecules with high potential for cancer treatments without compromising other metrics. When calculating molecular similarity to existing anti-cancer therapeutics, the top molecules generated are chemically similar, which means that TAIGA did manage to learn some understanding on what makes a drug anti-cancer. The inventors also see TAIGA's manages to generate a high amount of novel and diverse molecules while maintaining a high validity rate. Looking at the other metrics, such as QED or SAS, the inventors can see that TAIGA can generate anti-cancer molecules while maintaining around the same SAS score but having lower QED scores. When looking at anti-cancer molecules, some of them violate Lipinski's rule of 5, so it makes sense to have lower QED (which as part of its average uses properties from the rule of 5) scores as a trade-off for anti-cancer activity.

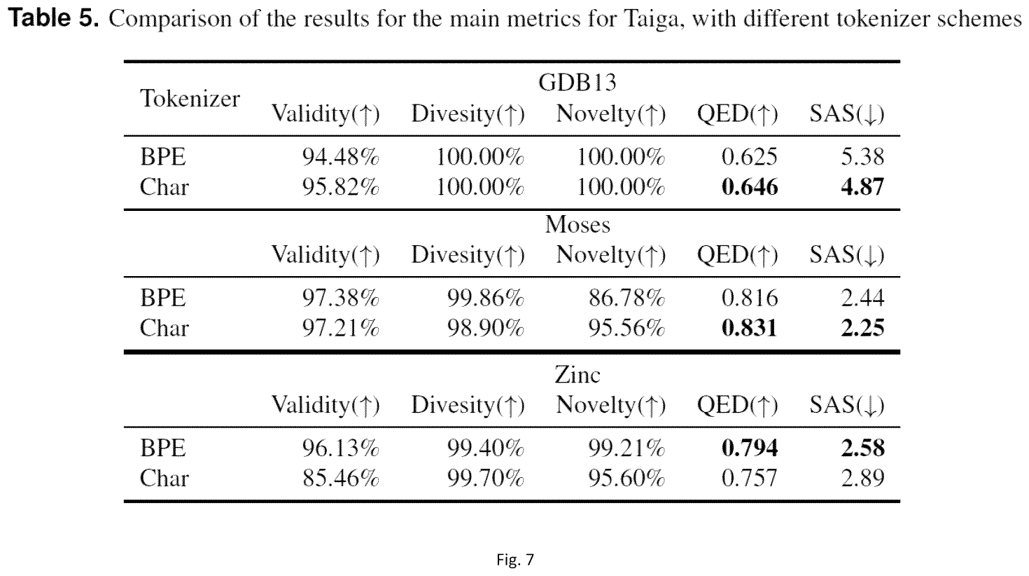

Reference is also made to FIG. 7, which depicts a table (Table 5). Table 5 shows a comparison of results for the main metrics for TAIGA, with and without the RL stage, as experimentally obtained by embodiments of the invention.

The inventors conducted an ablation study to evaluate the contribution of the RL stage on TAIGA's performance. As can be seen in the results presented in Table 5, with the Moses dataset, before the RL stage TAIGA underperformed in terms of validity and novelty when generating molecules; in addition, the QED value obtained was similar to the mean QED of the dataset (see Table 1). However, after the RL stage, the model was able to find a policy that enables better maximization of the QED. This is demonstrated by the increase in the mean QED of the molecules generated and the increase in the validity and novelty scores.

Reference is also made to FIG. 8, which is a table (Table 6), presenting cross-dataset novelty scores of the molecules generated, as experimentally obtained by embodiments of the invention.

The inventors can see a similar phenomenon with the GDB dataset when comparing the results before and after the RL stage. Although the model was able to generate more valid molecules before the RL stage, the difference in the mean QED value obtained before and after the RL stage emphasizes the fact that the model was able to learn how to generate highly optimized molecules with just a slight trade-off in terms of the validity. A similar improvement was seen with the Zinc dataset; before the RL stage, TAIGA struggled to generate valid molecules and obtained a mean QED similar to that of the dataset itself. After the RL stage, TAIGA generated more than 15% more valid molecules without significantly compromising the performance in terms of the diversity and novelty metrics; its mean QED also improved.

Another setting the inventors tested is using RL directly without incorporating the language modelling task first. The results are not included in the table since the model wasn't able to converge at all. After the first 20 steps it reached a point where the it failed to generate valid molecules (or any molecular formula at all). To further assess TAIGA's ability to generate novel molecules and not overfit to the training data, for each dataset the inventors calculated the novelty scores of the molecules generated in Section based on the other datasets. As seen in Table 6, TAIGA generated novel and unseen molecules that do not exist in the other datasets. This reinforces the idea that existing difficulties with SMILES strings can be overcome by combining transformers with RL.

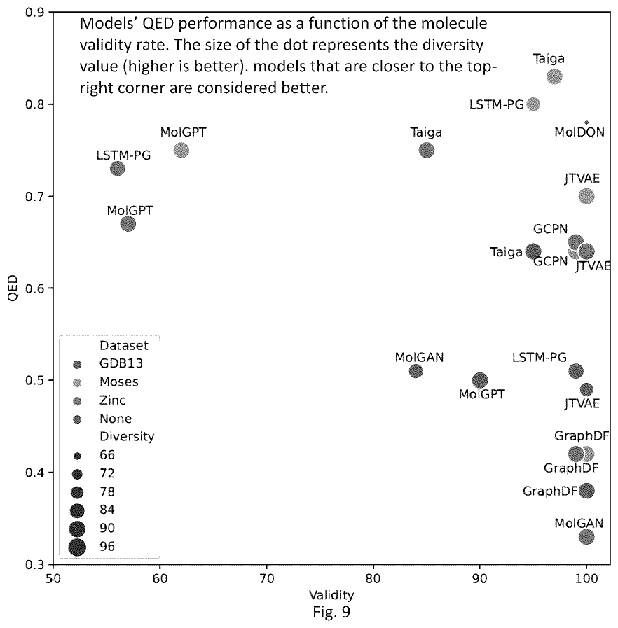

Reference is now made to FIG. 9, which is a chart showing experimental results of TAIGA's QED performance as a function of molecule validity rate. The size of the dot represents the diversity value (bigger is better). Models that are closer to the top-right corner may be considered better.

The inventors have proposed a solution for the de-novo drug design problem of generating novel molecules with desired properties. The inventors have introduced TAIGA, a transformer-based architecture for the generation of molecules with desired properties. TAIGA may use a two-stage, or two-level approach by first learning a language modelling task and then optimizing for the desired properties using RL.

The inventors' experimental results have demonstrated that the use of an attention mechanism may enable TAIGA to overcome the problem of generating invalid SMILES strings. When compared to an RNN using the same RL technique, TAIGA achieved similar or better results on all metrics across all of the examined datasets.

While all of the examined methods try to achieve the highest QED scores by directly optimizing for it on the generation task, TAIGA outperformed state-of-the-art methods when generating molecules with the highest QED scores and obtained similar or better results in terms of validity, novelty, and diversity when generating arbitrary molecules.

When optimizing for biological properties like pIC50, TAIGA reduced the concentration required by a factor of 3-5 for the same therapeutic effect (evaluated by an external property predictor), while maintaining similar scores on all other metrics, when using all datasets as baselines for training.

When optimizing for anti-cancer activity, TAIGA managed to achieve better anti-cancer activity while maintaining similar scores on all other metrics.

Additionally, when examined by expert pharmacologists, several of the top molecules generated by TAIGA evaluated as easily synthesizable and exhibited high probability of having anti-cancer properties, thus emphasising the advantage of the RL stage, which allows TAIGA to optimize properties that are not derived from the model itself.

The inventors' proposed method for molecule generation can enhance the drug development process by generating candidate molecules with improved therapeutic properties that are better than those of existing drugs on the market. The drug development process takes an average of 13 years, of which half are spent searching for lead molecules, and the proposed method can help reduce the time devoted to this task.

### Methods

The inventors define the molecule generation and optimization tasks similarly to the formulations used by the authors presenting GraphDF15. Given a set of molecules {mi}Mi=1 and a score function R(m)→R, the molecule generation task is defined as learning a generation model pθ (·), such that pθ (m) is the probability of generating molecule m. The optimization task is defined as maximizing EM˜pθ[R(m)] with respect to R (in the context of molecules, R can be the IC50 of the molecule or or any property one might want to maximize).

FIG. 1 illustrates the proposed method. TAIGA is based on a two-stage process in which the inventors first train a transformer-based architecture on a general language modelling task by having the model predict the next token in the sequence of SMILES strings. Then the inventors apply policy gradient RL, to achieve the desired molecular properties by learning a policy that maximizes the desired property as the reward in the RL stage. The main advantage of the proposed method is its utilization of a pretrained model capable of learning both the underlying rules of chemistry and the grammar of SMILES strings, which acts as an initial policy by training on the next-character prediction task. This improves the model when applying the RL algorithm.

Similar to MolGPT2, the inventors use a GPT-like decoder-based transformer model as an auto-regressive model for language modelling. The model consists of several decoder-only blocks stacked one after another. Each block uses the self-attention mechanism. This attention mechanism takes a set of keys, queries, and values (Q, K, V) as inputs, applies a dot product operation between the queries and the keys, and then computes the attention weights for the values by using the SoftMax function on the result of the dot product. The attention mechanism is formulated as in the equation below:

$$Attention ( Q , K , V ) = SoftMax ( QKT / d 0.5 ) V , \qquad Eq . 4$$

In order to learn different representations, the inventors use multi head attention (denoted herein as “MultiHead”), which may allow embodiments of the invention to attend to information for different positions at the same time as defined in equation Eq. 5 below:

$$MultiHead ( Q , K , V ) = Concat ( {head}_{1} , … , {head}_{n} ) {W}^{o} , \qquad Eq . 5$$

and where the attention of each head (head;) is defined by Eq. 6, below:

$${head}_{i} = Attention ( {QW}_{i}^{Q} , {KW}_{i}^{K} , {VW}_{i}^{V} ) , \qquad Eq . 6$$

where W^{Q}ᵢ, W^{K}ᵢ, W^{V}ᵢ are the projection matrices of head i.

To train a model that can carry out text generation tasks, the inventors have masked future tokens to prevent tokens from attending consecutive tokens when computing the self-attention mechanism.

The inventors then defined a transformer decoder block in the equations below:

$${z}_{1} = {x}_{1 - 1} + MHA ( LayerNorm ( {x}_{1 - 1} ) ) , and {x}_{1} = {z}_{1} + MLP ( LayerNorm ( {z}_{1} ) ) , \qquad Eq . 7$$

where x₁₋₁ is the input from the previous block, MLP is a multi-layer feed-forward network and MHA is the multi head attention defined previously. The inventors can then stack as many layers of decoder blocks as required in order to create the model.

In this subsection, the inventors formulate the RL problem for molecule graph generation. The inventors define the SMILES generation as a Markov decision process M=(S,A,P,R,γ).

Observation Space: A single state is represented as a vector F, the inventors assume there is a finite set of character types that can be used to represent a SMILES string bounded by n, in which F ∈ R¹ where fi belongs to {0, . . . , n}. S={sᵢ} is the state space of all possible intermediate SMILES strings with length t≤T; T denotes the terminal state after the model generates an EOS token or reaches a maximal length; and s₀, which is the initial state, is an empty string.

Action Space: A={aᵢ} is the set of actions the agent can take. In this case, all of the possible actions are the characters in the vocabulary you can append to the SMILES representation of the molecule, so the inventors assume that ai belongs to {0, . . . , n}.

Transition Dynamics: P is the transition dynamics that specify the probability of reaching a certain state given the current state and the chosen action, p(sₜ₊₁|sₜ, aₜ), since the state and action space consist of only characters; the transition dynamics are simply p(sₜ₊₁|sₜ, aₜ)=1, since appending a character is deterministic.

Reward Function: R is the reward function for a given molecule. The inventors define the reward as zero for all intermediate states, R(sₜ)=0. R(s_{T})=f(s_{T}) is a function applied to the molecule generated, and γ is the discount factor.

The inventors can now define the task of finding the set of parameters for their transformer-based network which maximizes the expected reward of the objective function J(θ) as in the equation below:

$${max}_{θ} J ( θ ) = {\sum }_{s ∈ S} {d}^{π} ( s ) {V}^{π} ( s ) = {\sum }_{\vert s \vert = T} {d}^{π} ( s ) {V}^{π} ( s ) \qquad Eq . 8$$

where d^{π} is the state distribution and V^{π} is the value function. Since it is unreasonable to compute the sum of all terminal states, which are all of the states that end with the EOS token, due to the large number of terminal states, the inventors sample them. Based on the rule of large numbers, the inventors can approximate this sum. Then the inventors determine the gradient of the expected value using policy π_{θ}(a|s).

$${∇}_{θ} J ( θ ) , = {𝔼}_{π} [ {Q}^{π} ( s , a ) {∇}_{θ} ln {π}_{θ} ( a \vert s ) ] = {𝔼}_{π} [ {G}_{t} {∇}_{θ} ln {π}_{θ} ( a \vert s ) ] \qquad Eq . 9$$

where Gₜ is the return of the trajectory and is defined in the equation below:

$${G}_{t} = {R}_{t + 1} + γ {R}_{t + 2} + {γ}^{2} {R}_{t + 2} + … + {γ}^{T - 1} {R}_{T} \qquad Eq . 10$$

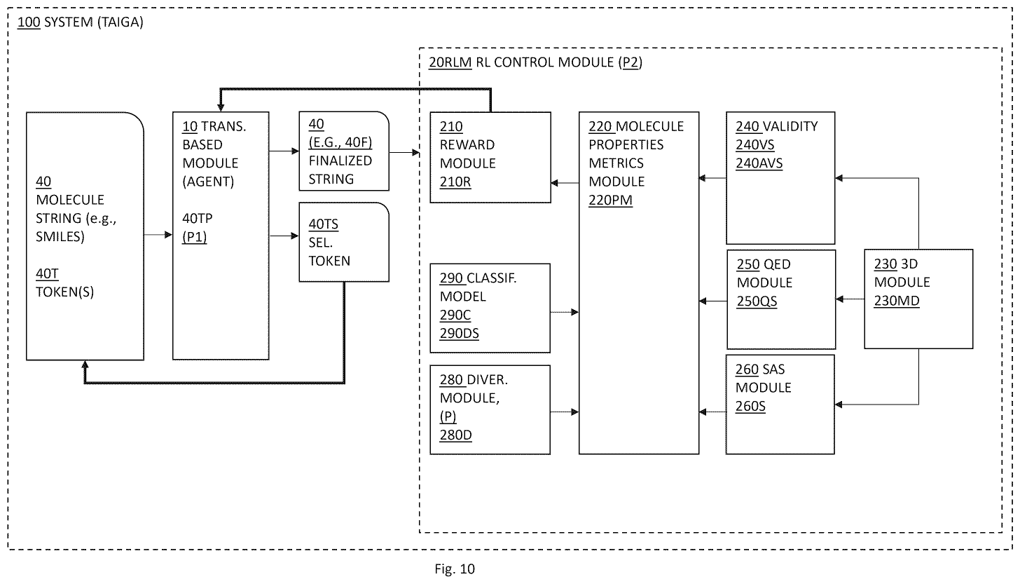

Reference is now made to FIG. 10, which depicts a system 100 (also referred to herein as “TAIGA”) for designing a drug, according to some embodiments of the invention. System 100 of FIG. 10 may be the same as system 100 of FIGS. 2A, 2B.

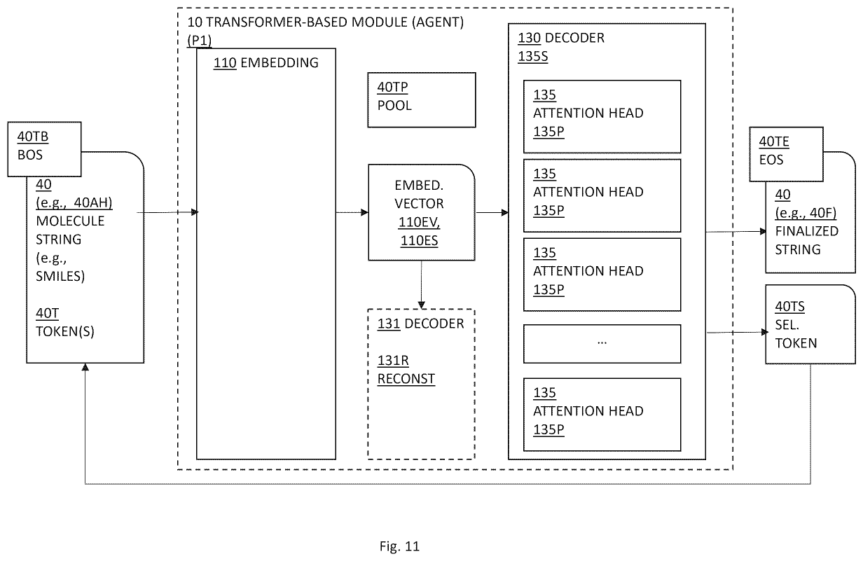

Reference is also made to FIG. 11, which depicts a transformer-based agent, that may be included in system 100 for designing a drug, according to some embodiments of the invention.

According to some embodiments of the invention, system 100 may be implemented as a software module, a hardware module, or any combination thereof. For example, system 100 may be, or may include a computing device such as element 1 of FIG. 1, and may be adapted to execute one or more modules of executable code (e.g., element 5 of FIG. 1) to design a drug, as further described herein.

As shown in FIGS. 10 and 11, arrows may represent flow of one or more data elements to and from system 100 and/or among modules or elements of system 100. Some arrows have been omitted in FIGS. 10 and 11 for the purpose of clarity.

As shown in FIG. 10, system 100 may implement an iterative, generative process to produce a molecule string 40 (e.g., a “SMILES” string), that may include a plurality of tokens 40T, and may exhibits predetermined, or desired molecular, and/or pharmaceutical properties.

The term “token” (40T) may be used to describe a component of molecule string 40. For example, a token 40T may include an indication of a beginning of a molecule string (also denoted herein BOS or 40TB). In another example, token 40T may include one or more components of the molecule, such as a chemical element (e.g., an atom). In another example, token 40T may include indication of a relation (e.g., a chemical bond) between components of the molecule. In yet another example, token 40T may include an indication of a beginning of a molecule string (also denoted herein EOS or 40TE).

As shown by the bold arrows of FIG. 10, the generative process may be iterative on two levels. A first (lower) level, of the iterative process may be referred to as a “transformer-based process” (denoted P1). The transformer-based process may include a plurality of iterations or repetitions, driven by a transformer-based module 10, also referred to herein as agent 10. Each iteration of the transformer-based process may include (a) transformer-based analysis of an ad-hoc structure of molecule string 40, (b) selection of a specific token (denoted “selected token” or 40TS) based on the transformer-based analysis, and (c) appendage of selected token 40TS to molecule string 40, to be re-analyzed. The transformer-based process may be repeated until an end condition is met, and the molecule string 40 is finalized by appending an EOS token 40TE at its end.

A second (upper) level of the generative process may be referred to herein as a Reinforced Learning (RL)-based process (denoted P2). As shown in FIG. 10, the RL-based process may be performed by an RL control module 20. The RL-based process may also be implemented as an iterative process that may include a plurality of iterations. Each iteration of the RL-based process may include (a) RL-based analysis of different, finalized molecule strings 40 (denoted 40F) to calculate a reward value 210R, and (b) providing the reward value as feedback to the transformer-based module (agent 10), to incrementally improve the selection of tokens 40T by the transformer-based process.

According to some embodiments, during the transformer-based process, agent 10 may receive an ad-hoc molecule string data element 40, representing ad-hoc structure of a molecule. The term “ad-hoc” may be used in this context to indicate a current composition of molecule string 40, that pertains to a current stage or iteration of the transformer-based process.

In an initial iteration of the transformer-based process, the ad-hoc molecule string 40 may contain at least one token 40T that is a “Beginning of String” (BOS) 40TB token, indicating a beginning of molecule string 40. Additionally, or alternatively, the ad-hoc molecule string 40 may include tokens 40T that represent one or more components (e.g., elements) of the represented molecule and/or one or more relations (e.g., chemical bonds) between the components of the represented molecule.

As shown in FIG. 11, agent 10 may include an embedding module 110, pretrained to apply an embedding algorithm on the ad-hoc molecule string 40, thereby obtaining an embedding vector 110EV. Embedding module 110 may be trained using language modeling loss, e.g., to predicting the next token 40T in the sequence from the current token and previous tokens 40T. Embedding vector 110EV may represent the content and/or structure of the ad-hoc molecule string 40 in an embedding space 110ES, having a reduced dimensionality (e.g., being shorted than molecule string 40). For example, embedding module 110 may be, or may include an encoder, pretrained in conjunction with a corresponding decoder module 131. In such embodiments, embedding module 110 may be adapted to produce one or more embedding vectors 110EV as low dimensionality representations of respective one or more incoming molecule string 40, whereas decoder module 131 may be pretrained to reconstruct the one or more incoming molecule strings 40 from the one or more embedding vectors 110EV.

Additionally, or alternatively, agent 10 may include at least one transformer-based decoder module 130 (also referred to herein as “generator” 130) which may, or may not be the same as decoder module 131. As elaborated herein decoder module 130 may include a plurality of attention heads 135.

As known in the art, transformer-based decoders or generators are a type of generative NN architecture that has gained popularity, e.g., in the field of Natural Language Processing (NLP). Transformer-based generators allow different parts of an input sequence (e.g., different words in a sentence) to be weighed differently when making predictions, thereby enabling the model to focus on relevant, inter-dependent information. Transformer-based generators may include one or more “attention heads”. Each attention head may be configured to calculate an “attention score”, pertaining to a specific location or element in a sequence (e.g., a word in a sentence), based on examination of its relationship with every other element in the sequence. These attention scores may then be used to create a weighted sum, or weighted score, for one or more candidate element in a pool of candidate elements. These weighted scores may, in turn, be used to select (also termed herein “predict”) a subsequent element in the sequence from the pool of elements.

For example, an ad-hoc sentence may include the string “Have a nice”. A pool of available candidate words may include the group {“dog”, “house”, and “day”}. Based on examination of each candidate word's relationship with every other word in the ad-hoc sentence, a transformer-based generator may apply interim scores to each of candidate word in the pool. These interim scores may represent a distribution of probability for each candidate word to be selected, based on a respective location (other word) in the ad-hoc sentence. The transformer-based generator may then apply weighted sums on the interim scores, to create a global score for each candidate word in the pool. The transformer-based generator may subsequently use the global scores to predict, or select a word from the pool of candidate words. For example, the transformer-based generator may select (predict) the word “day” to append to the ad-hoc sequence, thereby producing an updated sequence: “Have a nice day”.

According to some embodiments, each attention head 135 of the plurality of attention heads 135 may be pretrained to provide a distribution of probabilities 135P for selecting specific tokens 40T of the set, or pool 40TP of available tokens, based on different locations in the molecule string. Agent 10 (decoder 130) may then calculate an attention score 135S as a function (e.g., a weighted sum) of the plurality of probabilities 135P, and predict, or select a token 40T (40TS) based on the attention scores 135S.

For example, agent 10 may apply attention heads 135 of pretrained transformer-based decoder model 130 on embedding vector 110EV, to select a subsequent token 40T (denoted 40TS) from a predetermined set of tokens based on the distribution of probabilities 135P provided by the plurality of attention heads.

In the example of FIG. 2B (panel (a)), the pool or set 40TP of allowable, candidate tokens 40T may include, for example, symbols that represent chemical elements, numbers, and relationships, e.g., ‘C’ for Carbon, ‘O’ for Oxygen, ‘═’ for a dual chemical bond, etc. The series of selected tokens 40TS of transformer-based decoder module 130 represents a current state of the ad-hoc molecule string 40, and includes: {‘C’, ‘O’, ‘N’, ‘═’, ‘C’, ‘1’, ‘C’, ‘C’, ‘C’}. In this example, the symbol ‘(’ is the latest selected token 40TS, due to its superior attention score 135S (0.9) over all other candidate tokens (e.g., where score 135S of ‘C’ is 0.07, score 135S of ‘#’ is 0.03, etc.).

In each iteration of the transformer-based process, agent 10 (e.g., decoder module 130) may append the predicted, or selected token 40TS to the ad-hoc molecule string 40. This iterative process of selection and appendage of tokens 40T may proceed until agent 10 may detect occurrence of an end condition.

For example, agent 10 may identify an end condition as one where molecule string 40 has reached, or surpassed a predetermined length. In such a condition, agent 10 may append an EOS token at the end of molecule string 40. Agent 10 may thus indicate that molecule string 40 is finalized 40F, and determine the composition of molecule string 40 as a drug.

In another example, as shown in FIG. 2B (panel (b)), transformer-based generator (decoder) 130 may determine that an attention score 135S of an EOS token 40TE exceeds scores 135S of all other candidates in the set of optional tokens 40T. Decoder 130 may thereby select 40TS EOS token 40TE, and append it at the end of the ad-hoc molecule string 40, thereby defining molecule string 40 as finalized 40F, and determining the composition of molecule string 40 as a candidate drug.

As shown in FIG. 10, system 100 may include a Reinforcement Learning (RL) control module 20RLM (or “module 20RLM” for short). Module 20RLM may be adapted to implement the iterative RL-based process as explained above. The iterative RL-based process is denoted herein as RL process P2.

According to some embodiments, in each iteration of process P2, module 20RLM may receive, from agent 10, at least one finalized molecule string 40F. RL module 20RLM may analyze finalized molecule string 40F as explained herein, to obtain a reward value 210R, associate with finalized molecule string 40F.

Module 20RLM may provide reward 210R as feedback to agent 40, which may, in turn, retrain decoder model 130 based on the obtained reward value.

For example, agent 10 may retrain the transformer-based decoder 130 by adjusting the distribution of probabilities 135P associated with one or more attention heads 135 of decoder 130, using policy gradients reinforcement learning based on the obtained reward value 210R.

Agent 10 may then reinvoke the generative, transformer-based iterative process P1, to produce another finalized molecule string 40F. Such iterations of iterative process P2 may repeat, until a predetermined condition is satisfied.

According to some embodiments, RL module 20RLM may include, or may be associated with a 3D model generator module 230 (or “3D module 230” for short), configured to receive a finalized molecule string (e.g., SMILES) 40, that represents chemical content of an underlying molecule, and calculate therefrom a 3D model 230MD representing a 3D structure of the underlying molecule. For example, 3D module 230 may employ a third-party application for cheminformatics and computational chemistry such as the currently “RDKit” application, to produce 3D model 230MD.

As shown in FIG. 10, RL module 20RLM may include a molecule property module 220, adapted to apply one or more processes of analysis on 3D model 230MD, to obtain values of one or more respective metrics or properties 220P of the 3D modelled 230MD molecule.

For example, RL module 20RLM may include a validity analysis module 240, configured to calculate a molecule property 220P that is a validity score 240VS representing validity of the 3D modelled molecule 230MD. In other words, validity analysis module 240 may apply a validation algorithm 240 on 3D model 230MD, to obtain a molecule-specific validity score 240VS of the underlying molecule, and wherein the metric of molecule property 220P may include the molecule-specific validity score 240VS.

For example, validity analysis module 240 may assess physicochemical properties such as molecular weight, partition coefficient, solubility, polar surface area, and the like. Additionally, or alternatively, validity analysis module 240 may evaluate the reactivity and stability of the 3D modelled molecule 230MD. validity analysis module 240 may compare the assessed physicochemical properties, and/or the evaluated reactivity and stability to predetermined thresholds, and determine the value of validity score 240VS according to the comparison (e.g., as a binary value: Valid/Invalid).

Additionally, or alternatively, validity analysis module 240 may apply the validation algorithm on a plurality of 3D models 230MD, originating from a respective plurality of finalized molecule strings 40F, to obtain a respective plurality of molecule-specific validity scores 240VS. Based on the plurality of molecule-specific validity scores, validity analysis module 240 may calculate a molecule property metric value 220 that is an agent validity score 240AVS, which may represent a percentage of valid finalized molecule strings from the plurality of finalized molecule strings.

In another example, RL module 20RLM may include a Quantitative Estimate of Drug-likeness (QED) analysis module 240. QED analysis module 240 may be configured to apply a QED analysis algorithm, calculate a molecule property 220P that is a molecule-specific QED score 250QS. According to some embodiments, molecule-specific QED score 250QS may be a numerical value (e.g., between [0, 1]) that may be calculated based on the “Lipinski's rule of five”, to integrate several criteria of molecule properties, including for example, molecular weight, lipophilicity, hydrogen bond donors, and hydrogen bond acceptors. QED score 250QS may thereby provide indication regarding the 3D modelled molecule's 230MD expected behaviour in a human body.

In another example, RL module 20RLM may include a Synthetic Accessibility Score (SAS) analysis module 260. SAS analysis module 260 may be configured to apply an SAS analysis algorithm, to calculate a molecule property 220P that is a molecule-specific SAS score 250QS. SAS score 250QS may represent an estimation of synthesizability (e.g., how easy it is to synthesize) the 3D modelled molecule 230MD. For example, SAS analysis module 260 may divide the 3D modelled molecule 230MD into fragments or substructures, to scoring each fragments based on its complexity or difficulty in synthesis. Such scoring may consider factors such as the availability of starting materials, reaction types needed, synthetic steps required, and the like. SAS analysis module 260 may then combine the scores of individual fragments to obtain an overall SAS score 250QS for the molecule.

As elaborated herein, molecule property module 220 may employ additional modules of analysis to calculate additional scores and metrics for assessing properties 220P of the underlying molecule.

For example, RL module 20RLM may include a diversity calculation module 280 (“diversity module 280” for short). Diversity module 280 may collaborate with molecule property module 220, to control agent 10 so as to invoke generative process P1 a plurality of times, to obtain a respective plurality (P) of finalized molecule strings 40F.

Based on the member tokens 40T of the plurality (P) of finalized molecule strings 40F, diversity module 280 may calculate a metric value of molecule properties 220P which is a molecule diversity score 280D.

Molecule diversity score 280D may represent a diversity among the plurality of finalized molecule strings 40F. For example, molecule diversity score 280D may include a count, or percentage of unique finalized molecule strings 40F from the plurality (P) of finalized molecule strings 40F. Additionally, or alternatively, molecule diversity score 280D may represent a mean of a user-defined similarity metric value among a plurality (e.g., all) pairs of finalized molecule strings 40F.

According to some embodiments, RL module 20RLM may include a molecule classification model 290 (or “classifier 290” for short), configured to classify a candidate molecule, either in the finalized molecule strings 40F (e.g., SMILES) for or in the 3D model 230MD form according to a one of predetermined classes.

For example, classifier 290 may include a third party ML-based model, adapted for molecular property prediction, such as the currently available “Chemprop” deep-learning model. As known in the art, molecular property prediction models may leverage deep neural networks such as graph neural networks (GNNs), for predicting various, predetermined molecular properties of interest 290C. Such molecular properties 290C may include, for example toxicity, solubility, bioactivity, efficacy of treatment of a specific disease, and the like.

According to some embodiments, RL module 20RLM may (e.g., during a preliminary training stage) train molecular property prediction models on labeled dataset 290DS, to learn the relationships between molecular structures and target properties. For example, dataset 290DS may include a plurality of annotated, or labeled data elements such as finalized molecule strings 40F and/or molecule models 230MD. Data elements 40F/230MD of dataset 290DS may be annotated in a sense that they may be associated with respective annotations or labels, which may indicate a value of the molecular property of interest 290C (e.g., value of efficacy 290C). As known in the art, system 100 may subsequently utilize a training scheme (e.g., a backward propagation scheme), to train classification model 290, while using training dataset 290DS as supervisory information.

Additionally, or alternatively, RL module 20RLM may (e.g., during a subsequent inference stage) apply, or infer pretrained ML-based classifier 290 on the finalized molecule string, to predict a value of a molecular property of interest 290C, such as efficacy 290C of a respective molecule in treatment of a predetermined medical condition (e.g., a specific type of cancer). Additionally, or alternatively, RL module 20RLM may apply, or infer pretrained ML-based classifier 290 on model 230MD, to predict the molecular property of interest 290C (e.g., value of efficacy 290C).

According to some embodiments, a reward module 210 of RL module 20 may be configured to calculate reward value 210R based on the one or more metrics of molecule properties 220P (e.g., 240VS, 250QS, 260S, 280D). In other words, reward value 210R may be a data element that may include one or more numerical values, indicating a level of performance, or “content” with molecule properties 220P of one or more finalized molecule strings 40F, in effort to refine or improve finalized molecule strings 40F according to predefined criteria.

For example, reward value 210R may include a weighted sum, or average over all metrics of molecule properties 220P (e.g., 240VS, 250QS, 260S, 280D).

In another example, a low (e.g., ‘Invalid’) value of a validity score 240VS may serve as negative feedback for agent 10, whereas a high (e.g., ‘Valid’) value may serve as positive feedback.

In another example, a low (e.g., ‘0.1’) value of an agent validity score 240AVS may serve as negative feedback for agent 10, whereas a high (e.g., ‘0.9’) value may serve as positive feedback.

In another example, a low (e.g., ‘0.1’) value of a QED score 250QS may serve as negative feedback for agent 10, whereas a high (e.g., ‘0.9’) value may serve as positive feedback.

In another example, a low (e.g., ‘0.1’) value of a SAS score 260S may serve as negative feedback for agent 10, whereas a high (e.g., ‘0.9’) value may serve as positive feedback.

In another example, a low (e.g., ‘0.1’) value of a diversity score 280D may serve as negative feedback for agent 10, whereas a high (e.g., ‘0.9’) value may serve as positive feedback.

In yet another example, reward module 210 may calculate reward value 210D based on a predicted property of interest (e.g., drug efficacy value) 290C, where a low (e.g., ‘0.1’) value of efficacy value 290C may serve as negative feedback for agent 10, and a high (e.g., ‘0.9’) value may serve as positive feedback.

It may be appreciated that any appropriate combination or integration (e.g., a weighted sum) of the above-mentioned forms of feedback may be applied according to specific implementation requirements.

Agent 10 may then retrain transformers 135 of decoder model 130 based on the obtained reward value 210R, and subsequently reinvoke the generative process P1, to produce one or more new, refined molecule strings 40. The new molecule strings 40 may be regarded as refined, in a sense that they may be based on the analysis of previous molecule strings 40 and subsequent, accumulated retraining of transformers 135.

As elaborated herein, iterations of the iterative RL process P2 and generative process P1 may repeat until a predetermined condition on the one or more metrics of molecule properties 220P is satisfied.

In other words, iterative RL process P2 may continue until some threshold of molecule properties 220P is achieved, or by limiting the number of training steps. For example, diversity score 280D indicates a satisfactory level of diversity, and agent validity score 240AVS indicates a sufficient portion of validity among the generated finalized molecule strings 40F.

In another example, iterative generative process PI may repeat until efficacy value 290C indicates reaching a sufficient number of potent, candidate drugs.

In another example, iterative generative process P1 may repeat until scores 240VS and 260S indicate reaching a satisfactory number of valid, synthetically feasible candidates.

It may be appreciated that other appropriate combinations of indicators and properties 220P may serve to determine that system 100 has acquired a satisfactory number of candidate finalized molecule strings 40F for drug synthesis, and that the generative process may be halted.

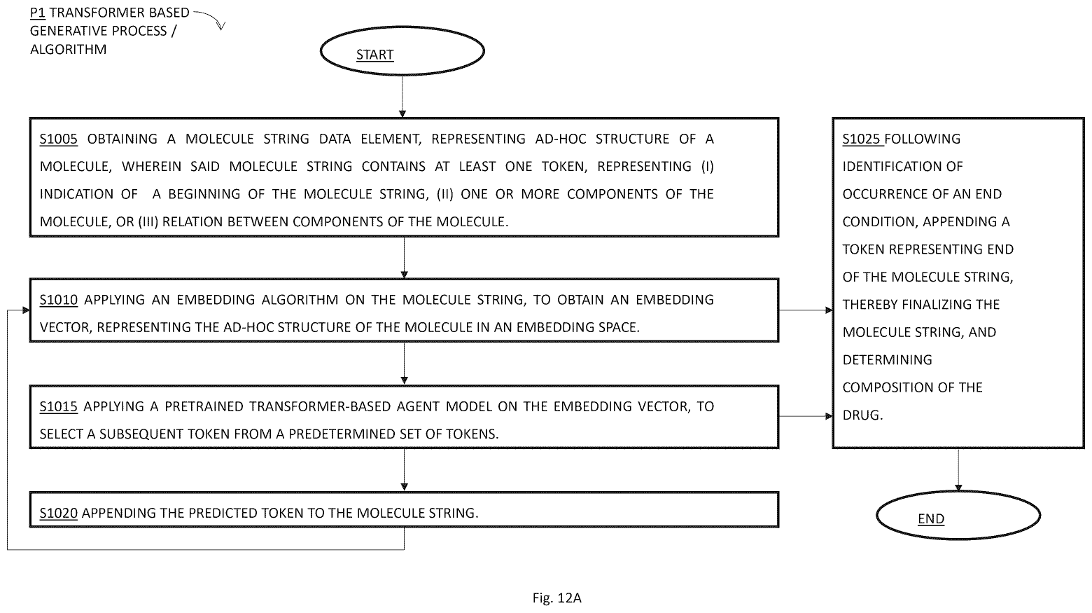

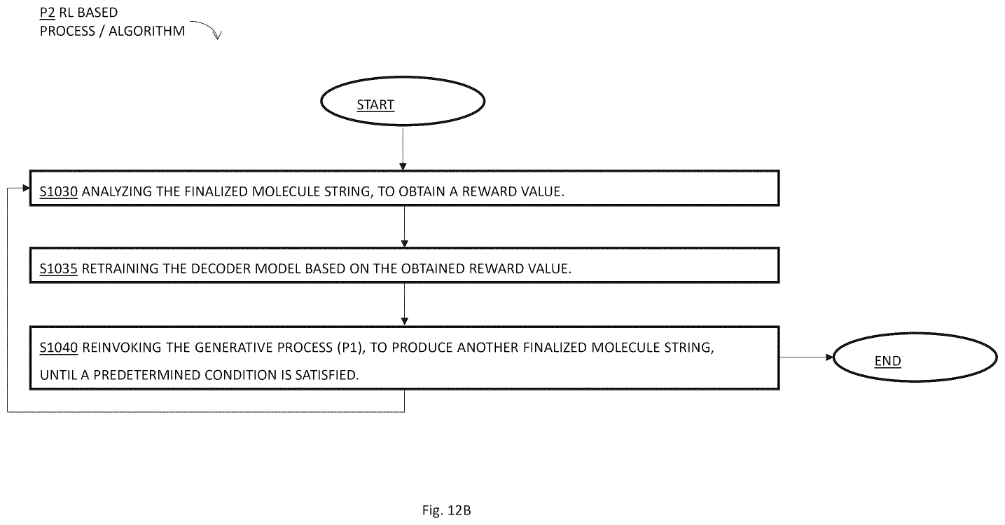

Reference is now made to FIGS. 12A and 12B which jointly depict a flow diagram of a method of designing a drug by at least one processor (e.g., processor 2 of FIG. 1), according to some embodiments of the invention. FIG. 2A depicts a first level of the method, that is referred to herein as an iterative, transformer-based generative algorithm (P1). FIG. 2B depicts a second (e.g., upper) level of the method, that is referred to herein as an iterative, RL-based algorithm (P2).

As shown in step S1005, the at least one processor 2 may obtain a molecule string data element (e.g., element 40 of FIG. 10), representing an ad-hoc structure of a molecule. Molecule string 40 may include at least one token (e.g., element 40T of FIG. 10). Token 40T may represent at least one of: (i) indication of a beginning of the molecule string (e.g., 40TB BOS of FIG. 11), (ii) one or more components of the molecule, and/or (iii) a relation (e.g., a chemical relation) between components of the represented molecule.

As shown in step S1010, the at least one processor 2 may employ an encoder, or an embedding module (e.g., 110 of FIG. 11) to apply an embedding algorithm on the molecule string, thereby obtaining an embedding vector (e.g., 110EV of FIG. 11). Embedding vector 110EV may represent the ad-hoc structure (e.g., 40) of the molecule in an embedding space.

As shown in step S1015, the at least one processor 2 may apply a pretrained transformer-based generative module (also referred to herein as a decoder, e.g., 130 of FIG. 11) on the embedding vector, to select 40TS a subsequent token 40T from a predetermined set of tokens.

As shown in step S1020, the at least one processor 2 may append the predicted or selected token 40TS to the molecule string 40. This iterative process may continue until occurrence of an end condition, as elaborated herein. Following identification of occurrence of an end condition (step S1025), the at least one processor 2 may append a token representing end of the molecule string (e.g., 40TE EOS of FIG. 11), thereby finalizing the molecule string 40 (now 40F), and determining composition of the drug.

As shown in FIG. 12B, the iterative transformer-based generative algorithm P1 or process may be embedded within an RL-based control algorithm or process P2.

For example, In step S1030, the at least one processor 2 may analyze the finalized molecule string 40F to obtain a reward value (e.g., 210R of FIG. 10), and may use reward value 210 as feedback into the transformer-based generative algorithm P1, e.g., to retrain the decoder model 130 (step S1035) based on the obtained reward value 210R.

As shown in step S1040, the at least one processor 2 may then reinvoke the generative process P1, to produce another finalized molecule string 40F. The iterative process P2 may continue until a predetermined condition is satisfied, e.g., when a sufficient number of finalized valid molecule strings 40F as generated, as elaborated herein.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method of designing a drug by at least one processor, the method comprising an iterative, generative process, wherein each iteration comprises:
obtaining a molecule string data element, representing ad-hoc structure of a molecule, wherein said molecule string contains at least one token, representing (i) indication of a beginning of the molecule string, (ii) one or more components of the molecule, or (iii) relation between components of the molecule;
applying an embedding algorithm on the molecule string, to obtain an embedding vector, representing the ad-hoc structure of the molecule in an embedding space;
applying a pretrained transformer-based decoder model on the embedding vector, to select a subsequent token from a predetermined set of tokens;
appending the predicted token to the molecule string; and
following identification of occurrence of an end condition, appending a token representing end of the molecule string, thereby finalizing the molecule string, and determining composition of the drug.

2. The method of claim 1, further comprising an iterative Reinforcement Learning (RL) process, wherein each iteration comprises:
analyzing the finalized molecule string, to obtain a reward value;
retraining the decoder model based on the obtained reward value; and
reinvoking the generative process, to produce another finalized molecule string, until a predetermined condition is satisfied.

3. The method of claim 2, wherein analyzing the finalized molecule string comprises:
based on the finalized molecule string, calculating a 3-Dimensional (3D) model representing a 3D structure of an underlying molecule;
analyzing the 3D model to obtain values of one or more metrics of molecule properties; and
calculating the reward value based on the one or more metrics of molecule properties,
and wherein the generative process is reinvoked until a predetermined condition on the one or more metrics of molecule properties is satisfied.

4. The method of claim 3, wherein analyzing the 3D model comprises applying a validation algorithm on the 3D model, to obtain a molecule-specific validity score of the underlying molecule, and wherein the metric of molecule properties comprises the molecule-specific validity score.

5. The method of claim 4, wherein analyzing the 3D model comprises:
applying the validation algorithm on a plurality of 3D models, originating from a respective plurality of finalized molecule strings, to obtain a respective plurality of molecule-specific validity scores; and
based on the plurality of molecule-specific validity scores, calculating an agent validity score, representing a percentage of valid finalized molecule strings from the plurality of finalized molecule strings,
and wherein the metric of molecule properties comprises the agent validity score.

6. The method of claim 3, wherein analyzing the 3D model comprises applying a Quantitative Estimation of Drug-Likeness (QED) algorithm on the 3D model, to obtain a molecule-specific QED score of the underlying molecule, and wherein the metric of molecule properties comprises the molecule-specific QED score.

7. The method of claim 3, wherein analyzing the 3D model comprises applying a Synthetic Accessibility Score (SAS) algorithm on the 3D model, to obtain a molecule-specific SAS score of the underlying molecule, and wherein the metric of molecule properties comprises the molecule-specific SAS score.

8. The method of claim 3, wherein analyzing the 3D model comprises:
invoking the generative process a plurality of times, to obtain a plurality of finalized molecule strings; and
based on the member tokens of the plurality of finalized molecule strings, calculating a molecule diversity score, representing a diversity among the plurality of finalized molecule strings,
wherein the metric of molecule properties comprises the molecule diversity score.

9. The method of claim 2, wherein analyzing the finalized molecule string comprises:
applying a pretrained Machine Learning (ML) based classification model on the finalized molecule string, to predict a value of efficacy of a respective molecule in treatment of a predetermined medical condition; and
calculating the reward value based on the predicted efficacy value,
and wherein the generative process is reinvoked until a predetermined condition on the predicted efficacy value is satisfied.

10. The method of claim 2, wherein the transformer-based decoder model comprises a plurality of attention heads, and wherein each attention head is pretrained to provide a distribution of probabilities for selecting specific tokens of the set of tokens, based on different locations in the molecule string.

11. The method of claim 10, wherein the decoder model is configured to select the subsequent token based on the distribution of probabilities provided by the plurality of attention heads, and wherein retraining the decoder model comprises adjusting the distribution of probabilities based on the obtained reward value.

12. A system for designing a drug, the system comprising: a non-transitory memory device, wherein modules of instruction code are stored, and at least one processor associated with the memory device, and configured to execute the modules of instruction code, whereupon execution of said modules of instruction code, the at least one processor is configured to:
obtain a molecule string data element, representing ad-hoc structure of a molecule, wherein said molecule string contains at least one token, representing (i) indication of a beginning of the molecule string, (ii) one or more components of the molecule, or (iii) relation between components of the molecule;
apply an embedding algorithm on the molecule string, to obtain an embedding vector, representing the ad-hoc structure of the molecule in an embedding space;
apply a pretrained transformer-based decoder model on the embedding vector, to select a subsequent token from a predetermined set of tokens;
append the predicted token to the molecule string; and
following identification of occurrence of an end condition, append a token representing end of the molecule string, thereby finalizing the molecule string and determining composition of the drug.

13. The system of claim 12, wherein the at least one processor is configured to apply an iterative RL algorithm on the generative process, wherein at each iteration of the RL algorithm the at least one processor is configured to:
analyze the finalized molecule string, to obtain a reward value;
retrain the decoder model based on the obtained reward value; and
reinvoke the generative process, to produce another finalized molecule string, until a predetermined condition is satisfied.

14. The system of claim 13, wherein the at least one processor is configured to analyze the finalized molecule string by:
based on the finalized molecule string, calculating a 3-Dimensional (3D) model representing a 3D structure of an underlying molecule;
analyzing the 3D model to obtain values of one or more metrics of molecule properties; and
calculating the reward value based on the one or more metrics of molecule properties,
and wherein the generative process is reinvoked until a predetermined condition on the one or more metrics of molecule properties is satisfied.

15. The system of claim 14, wherein the at least one processor is configured to analyze the 3D model by applying a validation algorithm on the 3D model, to obtain a molecule-specific validity score of the underlying molecule, and wherein the metric of molecule properties comprises the molecule-specific validity score.

16. The system of claim 15, wherein the at least one processor is configured to analyze the 3D model by:
applying the validation algorithm on a plurality of 3D models, originating from a respective plurality of finalized molecule strings, to obtain a respective plurality of molecule-specific validity scores; and
based on the plurality of molecule-specific validity scores, calculating an agent validity score, representing a percentage of valid finalized molecule strings from the plurality of finalized molecule strings,
and wherein the metric of molecule properties comprises the agent validity score.

17. The system of claim 14, wherein the at least one processor is configured to analyze the 3D model by applying a Quantitative Estimation of Drug-Likeness (QED) algorithm on the 3D model, to obtain a molecule-specific QED score of the underlying molecule, and wherein the metric of molecule properties comprises the molecule-specific QED score.

18. The system of claim 14, wherein the at least one processor is configured to analyze the 3D model by applying a Synthetic Accessibility Score (SAS) algorithm on the 3D model, to obtain a molecule-specific SAS score of the underlying molecule, and wherein the metric of molecule properties comprises the molecule-specific SAS score.

19. The system of claim 14, wherein the at least one processor is configured to analyze the 3D model by:
invoking the generative process a plurality of times, to obtain a plurality of finalized molecule strings; and
based on the member tokens of the plurality of finalized molecule strings, calculating a molecule diversity score, representing a diversity among the plurality of finalized molecule strings,
wherein the metric of molecule properties comprises the molecule diversity score.

20. The system of claim 13, wherein the at least one processor is configured to analyze the finalized molecule string by:
applying a pretrained Machine Learning (ML) based classification model on the finalized molecule string, to predict a value of efficacy of a respective molecule in treatment of a predetermined medical condition; and
calculating the reward value based on the predicted efficacy value,
and wherein the generative process is reinvoked until a predetermined condition on the predicted efficacy value is satisfied.

21. (canceled)

22. (canceled)
